(19) United States
(12) Patent Application Publication
(10) Pub. No.: US 2022/0111471 A1
(43) Pub. Date: Apr. 14, 2022

(54) USING X-RAY ANALYSIS DURING REFLOW THERMAL CYCLE FOR SOLDER JOINTS

(71) Applicant: LOON LLC., Mountain, CA (US)

(72) Inventors: Vineethkumar Nair, Mountain View, CA (US); Keith Fries, San Jose, CA (US)

(73) Assignee: LOON LLC., Mountain, CA (US)

(21) Appl. No.: 17/065,978

(22) Filed: Oct. 8, 2020

(51) Int. Cl. B23K 31/12; H05K 3/34; G01N 23/04; G01N 33/207; G01N 25/04; B23K 31/00; G06T 7/00

(52) U.S. Cl. CPC B23K 31/125; H05K 3/3494; G01N 23/04; G01N 33/207; B23K 2101/42; B23K 31/003; G06T 7/0004; H05K 2203/163; G01N 2223/629; G01N 25/04

(57) ABSTRACT

Aspects of the technology employ real-time monitoring and feedback for a printed circuit board fabrication system. An x-ray simulator can be used to aid in understanding metallurgical phase transformations in real time to ensure acceptable and reliable solder connections. This includes real-time data gathering and evaluation of solder-related printed circuit board data, including peak temperature, time above liquidus (TAL), ramp up and ramp down rates. Such information is used to identify the exact melting point and view specific soldering behavior in order to achieve an optimized soldering solution. This approach can provide effective solder joint analysis, which can reduce the likelihood of failure of a circuit board intended to operate in extreme environments for an extended period of time, such as the stratosphere.

### BACKGROUND

Fabrication of electronic circuits, such as components assembled on a printed circuit board (PCB), typically includes soldering of components onto the PCB or other substrate. Unfortunately, defects in the fabrication process can adversely impact component operation or render an entire electronics module inoperable, for instance due to poor electrical or thermal conductivity, or insufficient mechanical connectivity. Various types of defects relate to issues with solder joints, including cracking, cold joints, voids, head-in-pillow, and bridging between different contacts. Such defects may be caused by various manufacturing issues. For instance, a void in which the solder is substantially or entirely missing may occur due to different reasons, such as outgassing of flux in solder paste, vias in pads, or solder paste quality impacting its rheological properties.

Certain procedures, such as reflow under inert conditions, can be employed to reduce soldering-related defects. However, these procedures may be ineffective on their own when manufacturing circuitry that includes large or heavy components, especially components with strict alignment requirements. For example, it may also be difficult to detect voids beneath duplexers and other large components that may partially or completely block x-rays. Conventional manufacturing techniques can be insufficient when fabricating a communication module having duplexers or similar components. This issue can be compounded when the communication module is intended for use in extreme environments, such as the stratosphere, where very low temperatures and/or large swings in environmental conditions can accelerate system failures. By way of example, at stratospheric altitudes and temperature ranges, printed circuit board assemblies (PCBAs) can undergo physical strain, thereby causing solder joints to crack and create electrical open circuit conditions. These failures may be catastrophic, particularly when there is no feasible way to repair or replace the communication module when it is in the extreme environment.

### SUMMARY

Telecommunications connectivity via the Internet, cellular data networks and other systems is available in many parts of the world. However, there are many locations where such connectivity is unavailable, unreliable or subject to outages from natural disasters. Some systems may provide network access to remote locations or to locations with limited networking infrastructure via satellites or high altitude platforms (HAPs) located in the stratosphere. In the latter case, the communication equipment providing, e.g., LTE and/or 5G services, may be expected to operate for weeks, months or longer in a harsh environment without the possibility for repair should the equipment fail. Solder joint failures thus can adversely impact the ability of the platform to provide communication connectivity, which may necessitate launching of additional HAPs in a fleet in order to address a communication coverage deficiency.

One way to evaluate and address solder joint issues is as follows. On a printed circuit board without components (a bare board), thermocouples can be attached to selected points along the board where joint issues may be expected. The bare board can be run through an oven using a programmed reflow profile. The reflow data is then collected via the attached thermocouple locations. A defective board having one or more solder joint defects can then be run through the oven using the same thermal conditions and taken to x-ray inspection after cool down, in order to determine whether the defects have been remedied. However, in this approach, the visual evidence of the melting points and solder joint formation remains inaccessible, particularly when there are large components (e.g., multiplexers) or other devices that overlay the solder joints, such as ball grid arrays (BGAs), flip chip components, quad flat packs (QFPs) or Plastic Leaded Chip Carriers (PLCCs). Unfortunately, post-reflow inspection occurs too late to address defects not corrected during reflow.

In particular, the lack of real-time monitoring and feedback greatly limits the amount of useful data available needed for effective board fabrication. This significantly reduces the ability to improve and recover a defective solder connection. As a result, this can lead to suboptimal and/or less reliable solder connections, potentially leading to reduced electronic life and early failure due to multiple reworks and thermal cycles. For electronic assemblies operating in extreme conditions and subject to extreme temperatures and temperature changes including the stratosphere, a marginal solder joint can pose unacceptable risk and result in critical operational failure

Aspects of the technology employ real-time monitoring and feedback. For instance, an x-ray simulator can be used to aid in understanding metallurgical phase transformations in real time to ensure acceptable and reliable solder connections. This impacts the use of reliable electronic life in stratospheric and other conditions for functionality and operability.

According to one aspect, a reflow method is provided, which comprises loading a reflow profile into a processing system of a reflow module; installing a printed circuit board assembly within a reflow chamber of the reflow module, the reflow chamber including one or more temperature sensors arranged to detect temperature within the reflow chamber along one or more selected regions of interest of the printed circuit board assembly; arranging an x-ray inspection unit of the reflow module with respect to the reflow chamber, the x-ray inspection unit being configured to capture x-ray imagery of the printed circuit board assembly during reflow of solder; heating the printed circuit board assembly according to the reflow profile; during heating of the printed circuit board assembly, capturing x-ray imagery of selected areas of the printed circuit board assembly and capturing temperature data along the one or more selected regions of interest of the printed circuit board assembly; and analyzing the captured x-ray imagery and the captured temperature data to identify information about the solder during reflow.

In one example, the method further comprises modifying the reflow profile based on the analyzing; and heating another printed circuit board assembly according to the modified reflow profile. Modifying the reflow profile may include modifying one or more of peak temperature, overall reflow time, time above liquidus, ramp up rate, or ramp down rate.

In another example, analyzing the captured x-ray imagery and the captured temperature data is done in real time during reflow. In a further example, the information about the solder during reflow includes one or more of peak temperature, time above liquidus (TAL), ramp up rate or ramp down rates.

Information about the solder during reflow may include intermetallic connection thickness information for one or more solder joints of the printed circuit board assembly. In this case, when the intermetallic connection thickness information indicates a solder joint thickness below a threshold value, the method may include modifying the reflow profile. The method may further comprise evaluating the intermetallic connection thickness information in view of possible deformation under stratospheric conditions.

In another example, analyzing the captured x-ray imagery and the captured temperature data includes detecting a phase transformation point for the solder. In a further example, the method also includes selecting a different solder material or component alloy based on the analyzing. In yet another example, the x-ray imagery comprises still images or video imagery.

In one scenario, arranging the x-ray inspection unit of the reflow module with respect to the reflow chamber comprises placing the reflow module within the x-ray inspection unit. Here, the method may further comprise setting the x-ray inspection unit in vacuum after placing the reflow module within the x-ray inspection unit.

In another example, analyzing the captured x-ray imagery and the captured temperature data includes evaluating the captured x-ray imagery and the captured temperature to identify a point in time at which a phase transformation point of the solder occurs. In a further example, the method also comprises arranging the one or more temperature sensors to detect temperature within the reflow chamber along the one or more selected regions of interest of the printed circuit board assembly based on positions of one or more components on the printed circuit board assembly.

In yet another example, arranging the x-ray inspection unit includes orienting one or more x-ray imagers to capture the imagery along the one or more selected regions of interest of the printed circuit board assembly based on positions of one or more components on the printed circuit board assembly. And in a further example, analyzing the captured x-ray imagery and the captured temperature data to identify information about the solder during reflow includes identifying one or more solder defects along the printed circuit board assembly.

According to another aspect of the technology, a printed circuit board assembly may be fabricated using the reflow method described above. And according to a further aspect of the technology, a communication system comprises one or more printed circuit board assemblies fabricated by the method described above. Here, the communication system may be part of a high altitude platform configured to operate in the stratosphere.

### BRIEF DESCRIPTION OF THE DRAWINGS

FIG. 1 is a functional diagram of an example system in accordance with aspects of the technology.

FIG. 2 illustrates a balloon configuration in accordance with aspects of the technology.

FIG. 3 is an example payload arrangement in accordance with aspects of the technology.

FIG. 4 is an example x-ray image of a printed circuit board in accordance with aspects of the technology.

FIG. 5 illustrates an example of void formation beneath an electronic component of a printed circuit board.

FIGS. 6 A-B illustrates before and after placement of an electronic component on a printed circuit board in accordance with aspects of the technology.

FIG. 7 illustrates an example PBC with alignment fixtures in accordance with aspects of the technology.

FIG. 8 illustrates an angled x-ray of a PCB showing voids along different areas of the PCB.

FIG. 9 illustrates an example real time x-ray reflow simulation system in accordance with aspects of the technology.

FIG. 10 illustrates an example reflow module in accordance with aspects of the technology.

FIG. 11 illustrates a method in accordance with aspects of the technology.

### DETAILED DESCRIPTION

### Overview

The technology relates to processes for real-time data gathering and evaluation of solder-related printed circuit board data, including peak temperature, time above liquidus (TAL), ramp up and ramp down rates. This information is used to identify the exact melting point and view specific soldering behavior that would provide fabrication engineers detailed information to develop truly accurate processes for an optimized soldering solution. In the case of rework, such a tool would help to identify exact conditions wherein the solder re-melts and conditions itself to form good intermetallic connection. Such a system provides the opportunity to repair a PCBA in the fewest possible thermal cycles, which results in the least risk to reliability and functional life.

This approach can provide effective solder joint analysis, which can reduce the likelihood of failure of a PCBA intended to operate in extreme environments. For instance, ensuring an effective process may be mission critical for systems such as communication modules deployed on HAPs intended to operate in the stratosphere for extended periods of time. Electrical, thermal and/or mechanical failures of such components due to solder joint issues may render the entire communication system of a HAP inoperable or severely degraded.

Stratospheric HAPs, such as balloon-based HAPs, may have a float altitude of between about 50,000-120,000 feet above sea level. At such heights, the density of the air is very low compared to ground level. For example, while the pressure at ground level is around 1,000 mbar, the pressure in the lower stratosphere may be on the order of 100 mbar and the pressure in the upper stratosphere may be on the order of 1 mbar. The temperature in the stratosphere generally increases with altitude. For instance, in the lower stratosphere the average temperature may be on the order of −40° C. to −100° C. or colder, while the average temperature in the upper stratosphere may be on the order of −15° C. to −5° C. or warmer. In addition, while balloons and other HAPs in the stratosphere generally fly above the clouds and most weather conditions, the HAPs can be impacted by lightning-induced transients beneath them. Such environmental conditions can cause component or system-wide failures, which can reduce or cut short the HAP's operational lifetime. This may be especially true with voids impacting electronic circuitry.

The systems and processes discussed herein are configured to minimize the impact of solder joint issues on such circuitry. While these solutions are beneficial for PCB fabrication in general, including communication circuitry, they are particularly helpful for communication modules operating in extreme environments such as the stratosphere.

### Example Stratospheric Hap Systems

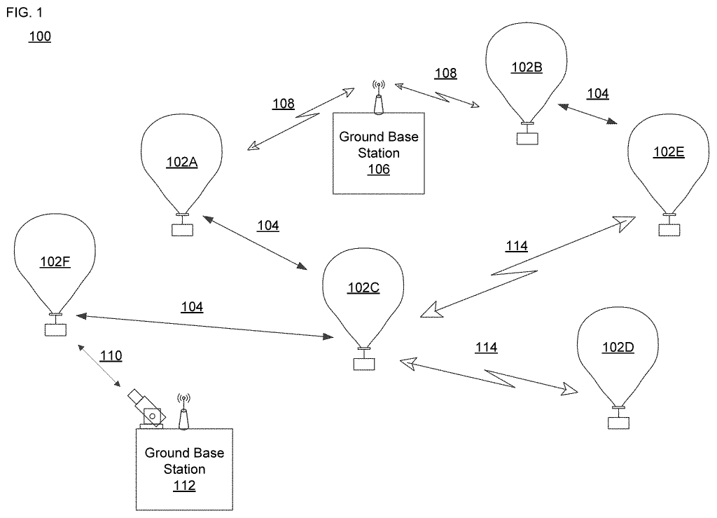

FIG. 1 depicts an example system 100 in which a fleet of balloons, airships or other HAPs described above may be used. This example should not be considered as limiting the scope of the disclosure or usefulness of the features described herein. System 100 may be considered a HAP network. In this example, network 100 includes a plurality of devices, such as balloons or other lighter-than-air (LTA) craft 102A-F as well as ground base stations 106 and 112. The network 100 may also include a plurality of additional devices, such as various computing devices (not shown) as discussed in more detail below or other systems that may participate in the network.

The devices in system 100 are configured to communicate with one another. As an example, the HAPs may include communication links 104 and/or 114 in order to facilitate intra-balloon communications. By way of example, links 114 may employ radio frequency (RF) signals (e.g., millimeter wave transmissions) while links 104 employ free-space optical transmission. Alternatively, all links may be RF, optical, or a hybrid that employs both RF and optical transmission. In this way balloons 102A-F may collectively function as a mesh network for data communications. At least some of the HAPs may be configured for communications with ground-based stations 106 and 112 via respective links 108 and 110, which may be RF and/or optical links.

In one scenario, a given balloon-type HAP 102 may be configured to transmit an optical signal via an optical link 104. Here, the given balloon 102 may use one or more high-power light-emitting diodes (LEDs) to transmit an optical signal. Alternatively, some or all of the balloons 102 may include laser systems for free-space optical communications over the optical links 104. Other types of free-space communication are possible. Further, in order to receive an optical signal from another balloon via an optical link 104, the balloon may include one or more optical receivers.

The HAPs may also utilize one or more of various RF air-interface protocols for communication with ground-based stations via respective communication links. For instance, some or all of balloons 102A-F may be configured to communicate with ground-based stations 106 and 112 via RF links 108 using various protocols described in IEEE 802.11 (including any of the IEEE 802.11 revisions), cellular protocols such as GSM, CDMA, UMTS, EV-DO, WiMAX, and/or LTE, 5G and/or one or more proprietary protocols developed for long distance communication, among other possibilities. The ground-based stations may include client devices such as mobile phones, computers, etc.

In some examples, the links may not provide a desired link capacity for HAP-to-ground communications. For instance, increased capacity may be desirable to provide backhaul links from a ground-based gateway. Accordingly, an example network may also include downlink balloons, which could provide a high-capacity air-ground link between the various HAPs of the network and the ground base stations. For example, in network 100, balloon 102F may be configured as a downlink balloon that directly communicates with station 112.

Like other HAPs in network 100, downlink balloon 102F may be operable for communication (e.g., RF or optical) with one or more other balloons via link(s) 104. Downlink balloon 102F may also be configured for free-space optical communication with ground-based station 112 via an optical link 110. Optical link 110 may therefore serve as a high-capacity link (as compared to an RF link 108) between the network 100 and the ground-based station 112. Downlink balloon 102F may additionally be operable for RF communication with ground-based stations 106. In other cases, downlink balloon 102F may only use an optical link for balloon-to-ground communications. Further, while the arrangement shown in FIG. 1 includes just one downlink balloon 102F, an example balloon network can also include multiple downlink balloons. On the other hand, a balloon network can also be implemented without any downlink balloons.

A downlink HAP may be equipped with a specialized, high bandwidth RF communication system for balloon-to-ground communications, instead of, or in addition to, a free-space optical communication system. The high bandwidth RF communication system may take the form of an ultra-wideband system, which may provide an RF link with substantially the same capacity as one of the optical links 104.

In a further example, some or all of HAPs 102A-F could be configured to establish a communication link with space-based satellites and/or other types of HAPs (e.g., drones, airplanes, airships, etc.) in addition to, or as an alternative to, a ground based communication link. In some embodiments, a balloon may communicate with a satellite or another high altitude platform via an optical or RF link. However, other types of communication arrangements are possible.

Each of the communication approaches noted above may employ one or more communication modules. As discussed further below, these modules may include duplexers, multiplexers and other components that may be significantly affected by voids created during the circuit board fabrication process.

The LTA platforms of FIG. 1 may be high-altitude balloons that are deployed in the stratosphere. As an example, in a high altitude balloon network, the balloons may generally be configured to operate at stratospheric altitudes, e.g., between 50,000 ft and 70,000 ft or more or less, in order to limit the balloons' exposure to high winds and interference with commercial airplane flights. In order for the balloons to provide reliable communication in the stratosphere, where winds may affect the locations of the various balloons in an asymmetrical manner, the balloons may be configured to move latitudinally and/or longitudinally relative to one another by adjusting their respective altitudes, such that the wind carries the respective balloons to the respectively desired locations. And as discussed below, lateral propulsion may also be employed to affect the balloon's path of travel.

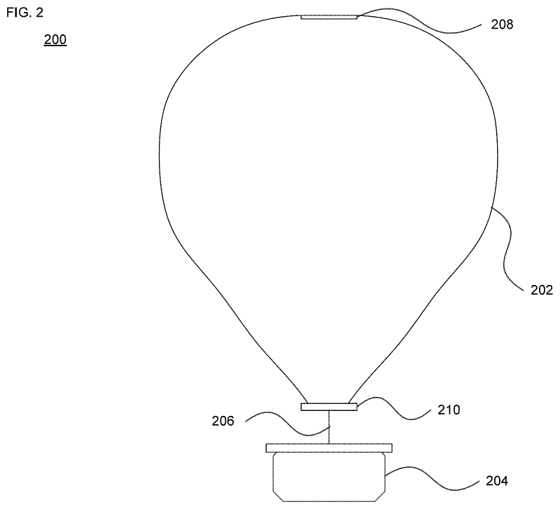

In an example configuration, a balloon-type HAP includes an envelope and a payload, along with various other components. FIG. 2 is one example of a high-altitude balloon 200, which may represent any of the balloon HAPs of FIG. 1. As shown, the example balloon 200 includes an envelope 202, a payload 204 and a coupling member (e.g., a down connect) 206 therebetween. At least one gore panel forms the envelope, which is configured to maintain pressurized lifting gas therein. For instance, the balloon may be a superpressure balloon. A top plate 208 may be disposed along an upper section of the envelope, while a base plate 210 may be disposed along a lower section of the envelope opposite the top place. In this example, the coupling member 206 connects the payload 204 with the base plate 210.

The envelope 202 may take various shapes and forms. For instance, the envelope 202 may be made of materials such as polyethylene, mylar, FEP, rubber, latex or other thin film materials or composite laminates of those materials with fiber reinforcements embedded inside or outside. Other materials or combinations thereof or laminations may also be employed to deliver required strength, gas barrier, RF and thermal properties. Furthermore, the shape and size of the envelope 202 may vary depending upon the particular implementation. Additionally, the envelope 202 may be filled with different types of gases, such as air, helium and/or hydrogen. Other types of gases, and combinations thereof, are possible as well. Shapes may include typical balloon shapes like spheres and “pumpkins”, or aerodynamic shapes that are symmetric, provide shaped lift, or are changeable in shape. Lift may come from lift gasses (e.g., helium, hydrogen), electrostatic charging of conductive surfaces, aerodynamic lift (wing shapes), air moving devices (propellers, flapping wings, electrostatic propulsion, etc.) or any hybrid combination of lifting techniques.

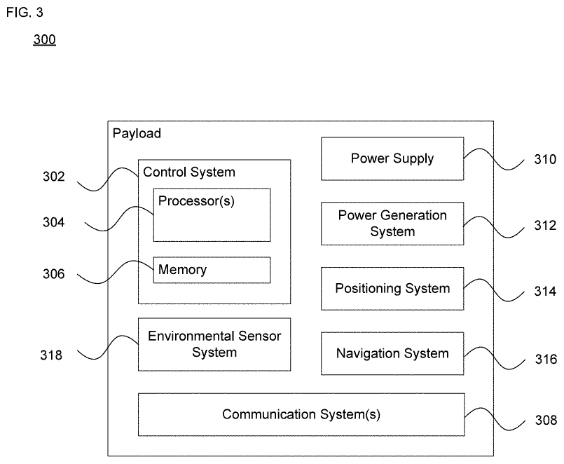

According to one example shown in FIG. 3, a payload 300 of a balloon or other HAP platform includes a control system 302 having one or more processors 304 and on-board data storage in the form of memory 306. Memory 306 stores information accessible by the processor(s) 304, including instructions that can be executed by the processors. The memory 306 also includes data that can be retrieved, manipulated or stored by the processor. The memory can be of any non-transitory type capable of storing information accessible by the processor, such as a hard-drive, memory card (e.g., thumb drive or SD card), ROM, RAM, and other types of write-capable, and read-only memories. The instructions can be any set of instructions to be executed directly, such as machine code, or indirectly, such as scripts, by the processor. In that regard, the terms “instructions,” “application,” “steps” and “programs” can be used interchangeably herein. The instructions can be stored in object code format for direct processing by the processor, or in any other computing device language including scripts or collections of independent source code modules that are interpreted on demand or compiled in advance. The data can be retrieved, stored or modified by the one or more processors 304 in accordance with the instructions.

The one or more processors 304 can include any conventional processors, such as a commercially available CPU. Alternatively, each processor can be a dedicated component such as an ASIC, controller, or other hardware-based processor. Although FIG. 3 functionally illustrates the processor(s) 304, memory 306, and other elements of control system 302 as being within the same block, the system can actually comprise multiple processors, computers, computing devices, and/or memories that may or may not be stored within the same physical housing. For example, the memory can be a hard drive or other storage media located in a housing different from that of control system 302. Accordingly, references to a processor, computer, computing device, or memory will be understood to include references to a collection of processors, computers, computing devices, or memories that may or may not operate in parallel.

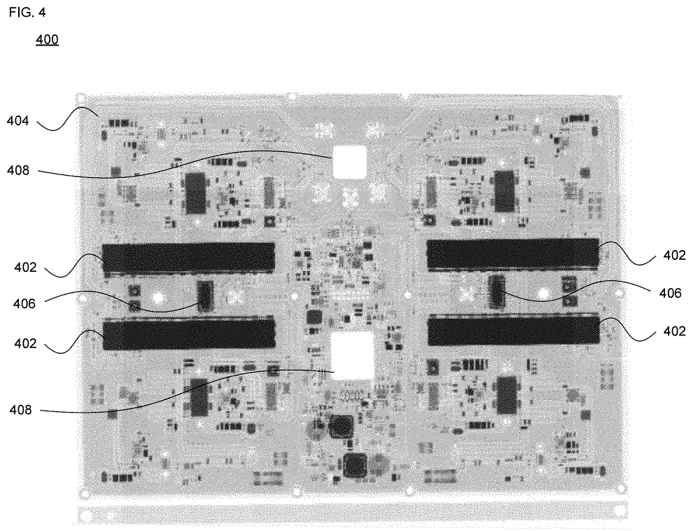

The payload 300 may also include various other types of equipment and systems to provide a number of different functions. For example, as shown the payload 300 includes one or more communication systems 308, which may transmit signals via RF and/or optical links as discussed above. The communication system(s) 308 include communication components such as one or more transmitters and receivers (or transceivers), one or more antennae, and a baseband processing subsystem (not shown). One or more duplexers (see FIG. 4) or other multiplexer components may be included in the communication system(s) 308.

The payload 300 is illustrated as also including a power supply 310 to supply power to the various components of the balloon or other HAP. The power supply 310 could include one or more rechargeable batteries or other energy storage systems like capacitors or regenerative fuel cells. In addition, the payload 300 may include a power generation system 312 in addition to or as part of the power supply. The power generation system 312 may include solar panels, stored energy (hot air), relative wind power generation, or differential atmospheric charging (not shown), or any combination thereof, and could be used to generate power that charges and/or is distributed by the power supply 310.

The payload 300 may additionally include a positioning system 314. The positioning system 314 could include, for example, a global positioning system (GPS), an inertial navigation system, and/or a star-tracking system. The positioning system 314 may additionally or alternatively include various motion sensors (e.g., accelerometers, magnetometers, gyroscopes, and/or compasses). The positioning system 314 may additionally or alternatively include one or more video and/or still cameras, and/or various sensors for capturing environmental data. Some or all of the components and systems within payload 300 may be implemented in a radiosonde or other probe, which may be operable to measure, e.g., pressure, altitude, geographical position (latitude and longitude), temperature, relative humidity, and/or wind speed and/or wind direction, among other information.

Payload 300 may include a navigation system 316 separate from, or partially or fully incorporated into control system 302. The navigation system 316 may implement station-keeping functions for the HAP to maintain position within and/or move to a position in accordance with a desired communication coverage or other service requirement. In particular, the navigation system 316 may use wind data (e.g., from onboard and/or remote sensors) to determine altitudinal and/or lateral positional adjustments that result in the wind carrying the balloon in a desired direction and/or to a desired location. Lateral positional adjustments may also be handled directly by a lateral positioning system that is separate from the payload. Alternatively, the altitudinal and/or lateral adjustments may be computed by a central control location and transmitted by a ground based, air based, or satellite based system and communicated to the HAP. In other embodiments, specific HAPs may be configured to compute altitudinal and/or lateral adjustments for other HAPs and transmit the adjustment commands to those other HAPs.

An environmental sensor system 318 is also shown, which may encompass some or all of the probes and other sensors mentioned above. In addition, the environmental sensor system 318 includes other sensors configured to detect information associated with lightning and other environmental conditions.

### Example Circuit Board Fabrication Approaches

FIG. 4 illustrates an example 400 of an x-ray image of a printed circuit board (PCB), for instance which may be used in a communication module of a HAP (e.g., an LTA HAP) as described above. X-ray images may be taken in order to inspect individual solder joints. As seen in this example, a set of duplexers 402 are arranged on PCB 404. And in between adjacent pairs of duplexers 402 are mechanical relays 406. The duplexers 402 and relays 406 are very dark in the image, indicating that they substantially or completely block x-rays. Other areas 408 of the PCB 404 may receive BGAs, flip chip components, QFPs and/or PLCCs. Thus, unless specialized equipment or particular angles are used, x-raying the PCB 404 will not indicate whether there are voids or other solder issues beneath such components. As a result, failures may only be detectable, in some instances, during RF calibration of the communication module. And by that time, it may be too late to address any defects from the fabrication process.

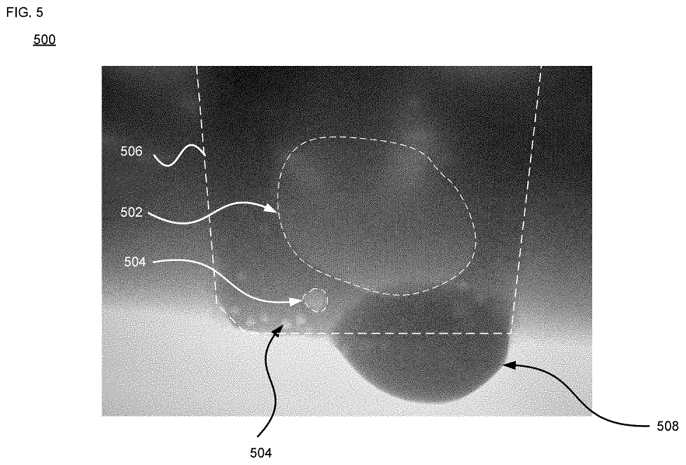

FIG. 5 illustrates an image 500 showing an example of voiding beneath a duplexer, such as any of the duplexers 402 of FIG. 4. Here, a large void 502 can be seen adjacent to a number of smaller voids 504 within a solder region 506 (bounded by dashed lines). Also shown is a solder ball 508 adjacent to the large void 502. In this example, the solder ball 508 was added after the solder region 506 was formed; however, it does not overcome potential issues involving the void 502.

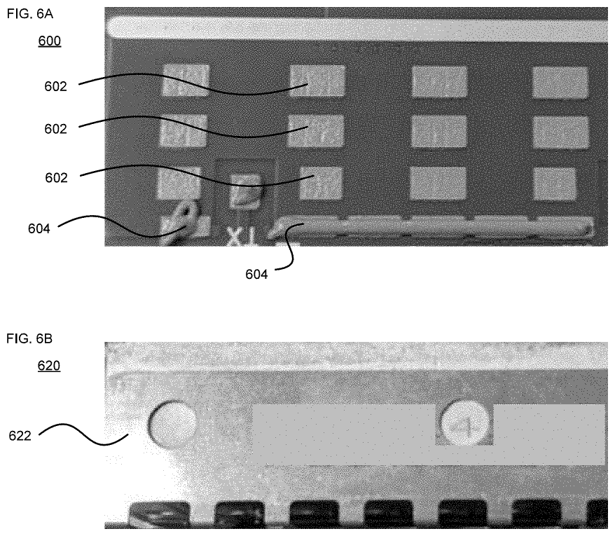

FIGS. 6A-B illustrate photographs of a portion of a PCB before and after a duplexer has been placed on it. As seen in view 600 of FIG. 6A, there are a series of pads 602 disposed along the PCB. These pads have solder already applied. Some pads have additional solder 604, as shown along the bottom left area of the image. The additional solder 604 may be added manually, which makes it difficult to ensure consistency of the amount of solder and repeatability in the specific placement of the additional solder. FIG. 6B illustrates view 620, in which the duplexer 622 has been placed on the pads 602. These pads cannot be readily inspected after the duplexer has been placed on them.

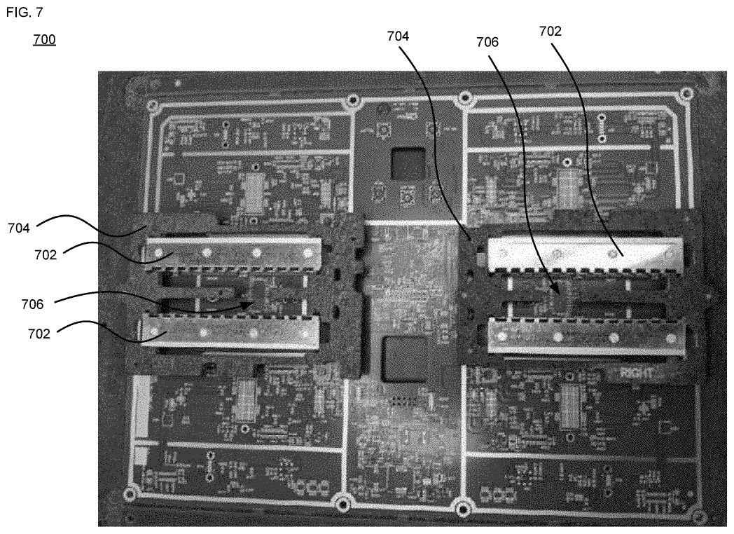

FIG. 7 illustrates a PCB 700 with duplexers 702 arranged over the pads, such as pads 602 of FIG. 6A. As can be seen, the duplexers 702 are large components taking up a substantial portion of the space along the PCB 700. The duplexers 702 can have strict alignment requirements and may be relatively heavy. By way of example, the duplexers 702 may be required to be aligned within +/−0.2 mm of the pad opening. Thus, as shown, alignment fixtures 704 may be required in order to ensure that the duplexers 702 maintain proper alignment. However, the alignment fixtures 704 may restrict airflow and retain heat, which can adversely impact the reflow profile during the fabrication process. The alignment fixtures 704 are removed after completion of the reflow thermal cycle that solders the duplexers to the PCB pads.

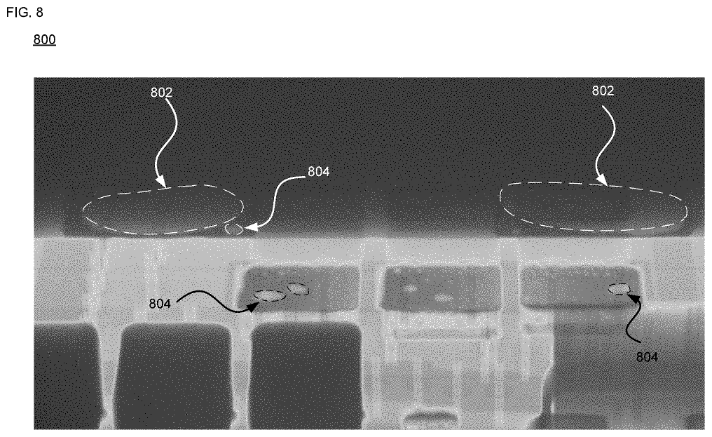

As seen by arrows 706, spaces for mechanical relays (not shown) are disposed between pairs of the duplexers 702. Another fabrication challenge is that the mechanical relays may be thermally sensitive. Even though a certain temperature profile may be desired in order to prevent void formation beneath the duplexers, this profile may be too hot for the mechanical relays, which can result in damage to the relays. In addition, manual addition of solder paste along edges of a duplexer, may also damage the adjacent relay and cause it to fail. FIG. 8 illustrates an angled x-ray view 800 of a sample PCB with large voids 802 and small voids 804 encircled by dashed lines for clarity.

As shown below, aspects of the technology evaluate the reflow process in real time using an x-ray reflow simulator. This is a heated stage that can hold and reflow the PCBA with soldered components while connected to an x-ray system, enabling a live x-ray view of PCBA solder joints. When performed in conjunction with measuring certain key indicators, metallurgical phase transformations can be determined in real time so that acceptable and reliable solder connections may be achieved for components that would otherwise obscure the solder connections. For instance, the system may gather critical data in real time, such as peak temperature, TAL, ramp up and ramp down rates, etc., as the solder begins to transition from solid to liquid phase. This information can be used to identify the exact melting point and to view particular soldering behavior. Such information helps to develop truly accurate processes for the optimized solution for fabricating the PCBA with the specific components. In the case of rework, such real time evaluation helps to identify precise conditions wherein the solder re-melts and conditions itself to form good intermetallic connections. This allows for corrections to be made to the solder without damaging the components through repeated reheating stages.

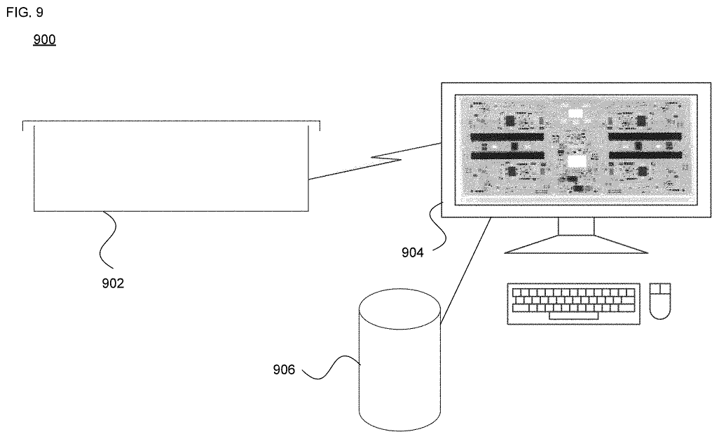

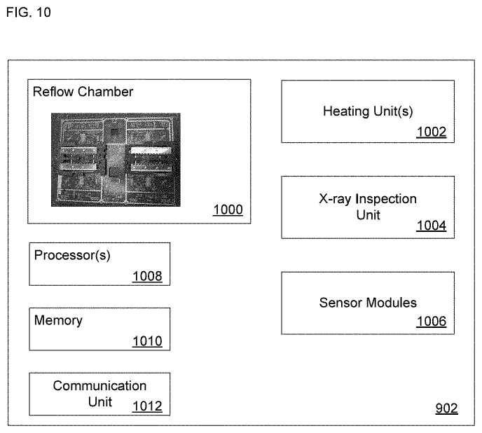

FIG. 9 illustrates an example arrangement 900 for a reflow system. As shown, the system includes a reflow module 902, a computer 904, and a database 906. The reflow module 902 is shown in FIG. 10 as having a reflow chamber 1000, one or more heating units 1002, an x-ray inspection unit 1004 and a set of sensor modules 1006. During operation, the reflow chamber 1000 may be placed within the x-ray inspection unit 1004, which may be set in a vacuum. The module 902 also includes one or more processors 1008, memory 1010 and a communication unit 1012, for instance to communicate with computer 904 and/or database 906.

The reflow chamber 1000 is configured to receive one or more printed circuit board assemblies. The heating unit(s) 1002 (e.g., ceramic or halogen heating modules) are configured to heat the reflow chamber 1000 to specific temperatures according to one or more temperature profiles stored in the memory 1010, for instance under the control of the one or more processors 1008. By way of example, the processor(s) 1008 may receive the temperature profile(s) from the computer 904 or database 906 via the communication unit 1012. Each profile may be based on the specific PCBA being fabricated, the types of surface mount components on the PCBA, and/or other factors such as soldering or circuit board manufacturing standards (e.g., IPC-A-610), for instance to produce PCBAs that are able to operate for extended periods of time (e.g., months or longer) in the stratosphere.

The reflow process within the reflow chamber may include control of the TAL to ensure that the solder flows as desired for the connections with different components. For instance, the ramp up rate may be no more than 1° C.-3° C./sec, and the TAL may be between 30 and 100 seconds. These factors may be impacted by the size of the PCBA, the characteristics of the selected components (e.g., duplexers v. flip-chips or BGAs), and other issues. Once the reflow process is finished and a determined temperature has been reached (e.g., room temperature, such as about 20° C.-22° C., or more or less), the PCBA may be removed from the reflow chamber. At this point, the PCBA may be routed through a series of inspections and measurements, such as automated optical inspection, X-ray inspection and quality control. If the PCBA meets the requisite standards (e.g., IPC standards) at these inspection stages, then the circuitry of PCBA is tested to confirm its functionality.

The x-ray inspection unit 1004 is arranged to provide real-time imagery, temperature and time information of the PCBA during the reflow process, and may have a field of view sufficient to view different layers or levels of the PCBA. For instance, it may be able to view different layers between 10-25 mm in depth and have an angular or oblique field of view (FOV) of between 30°-60°. Video and/or still imagery from the x-ray inspection unit 1004 may be stored in the memory 1010, or transmitted in real time via the communication unit 1012 to the computer 904 for display on a display device.

The sensor modules 1006 may include, by way of example, one or more temperature sensors and gas sensors (such as when an inert gas is used during reflow). This can include fixed and/or adjustable thermocouples disposed along the reflow chamber. There may be multiple temperature sensors dispersed along the reflow chamber, for instance located at 2-3 different corners of the chamber. Such sensors may be arranged at particular locations to ensure highly accurate temperature readings for the soldered areas of interest. The locations may be selected based on the types of components of interest. For instance, passive elements such as resistors do not need a thermocouple, while in contrast more complex packages such as BGAs, QFNs and through hole packages may benefit from thermocouples being placed as close as possible to the respective pads of those components.

In one scenario, the temperature sensors are able to make measurements continuously with a high level of granularity. By way of example, this can include making measurements every 0.01 s-0.05 s (or more or less), with an accuracy on the order of 0.1° C. (or more or less). This allows the system to measure peak temperature, TAL, ramp up and ramp down rates, etc., as the solder begins to transition from solid to liquid phase. The information from each sensor can be timestamped and associated with the specific sensor. Similarly, the video or still imagery from the x-ray inspection unit 1004 may also be timestamped with identification of the depth and/or FOV of the imagery. Such information may be stored in a database or other record in the memory 1010, and/or transmitted in real time to the computer 904 or the database 906.

The real time information is associated with an operating process window of intermetallic formations at the locations of interest. The intermetallic thicknesses determine the strength of solder joints and hence impacts the reliable life of operation. By way of example, at stratospheric conditions (e.g., stratospheric altitudes and/or temperature ranges) the PCBAs undergo deformation in directions that are difficult to predict. In such instances, locations with weaker intermetallic solder strength can fail much sooner than the rest of the assembly and stop functioning. Thus, it may be important to identify possible failure points with intermetallic thicknesses below a given threshold (e.g., X mm thick). If any failures due to reflow conditions are detected through X-ray analysis, such issues can be corrected by optimizing temperature and time in the reflow profile or through material (e.g., solder material) and/or alloy selection during the design phase for subsequent PCBA fabrication.

Returning to FIG. 9, the computer 904 may have all of the components normally used in connection with a personal computing device, including a one or more processors (e.g., a central processing unit (CPU), graphics processing units (GPUs) or other processing devices), memory (e.g., RAM and internal hard drives) storing data and instructions, a display (e.g., a monitor having a screen, a touch-screen, a projector, a television, or other device such as a smart watch display that is operable to display information), and user input devices (e.g., a mouse, keyboard, touchscreen or microphone). Moreover, although the computer 904 is depicted as a desktop-type computer, it may be any type of personal computing device, such as a laptop, netbook, tablet computer, etc.

Database 906 can be part of any type of storage system capable of maintaining information accessible by the computer 904, such as a hard-drive, memory card, ROM, RAM, DVD, CD-ROM, flash drive and/or tape drive. In addition, the storage system may be a distributed storage system where data is stored on a plurality of different storage devices which can be physically located at the same or different geographic locations. Database 906 may be connected to the computer 904 via a network (not shown) or may be directly connected to or incorporated into the computer 904.

Database 906 may store various types of information. For instance, it may receive real-time information from the reflow module 902, including x-ray imagery, temperature data and other sensor information. As noted above, such information may be timestamped, or otherwise indexed, so that the solder status along a specific portion of the PCBA can be determined at a particular point in the reflow process. The database may also store models or other temperature profile information, for instance based on past reflow evaluations for the same or similar PCBAs. In one scenario, the real-time x-ray capability of the present technology can be used to extract temperature and time information where solder re-melts and fixes known failures such as voids, cracks, open solder joints etc. This “fix” is captured by the phase transformation of solder, which may be detected manually by an operator and can vary from one reflow unit to another. When the system detects this phase transformation point, then it can collect the relevant information and use it to predict conditions for rework. Such information may be used as inputs that are fed back into the system to modify the reflow profile, such as the peak temperature, overall reflow time, time above liquidus (TAL), ramp up and ramp down rates, solder material or alloy type.

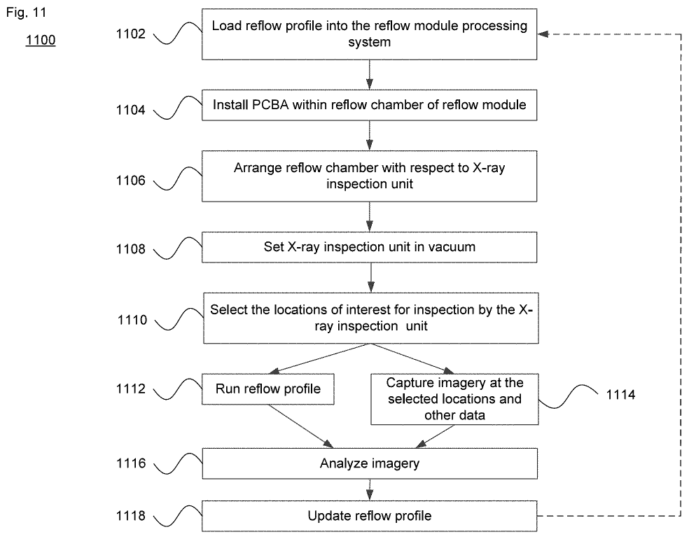

FIG. 11 illustrates a flow diagram 1100 for a reflow simulation method. At block 1102, a reflow profile is loaded into the processing system of the reflow module. This can include loading the reflow profile into memory 1010 and/or accessing the reflow profile by the processor(s) 1008. At block 1104, one or more PCBAs are loaded into the reflow chamber, e.g., chamber 1000 in FIG. 10. As noted above, there may be multiple temperature sensors dispersed along the reflow chamber near or otherwise adjacent to components of interest, for instance located at 2-3 different corners of the chamber. At block 1106, the reflow chamber is arranged with respect to an X-ray inspection unit, for instance by placing the chamber within the inspection unit. At block 1008 the inspection unit may be set in a vacuum. At block 1110, the locations of interest for inspection by the X-ray inspection unit are selected. This may be coordinated with placement of the temperature sensors along the reflow chamber. Then at block 1112 the reflow profile is run and concurrently, at block 1114, images and/or video (collectively, imagery) of the selected locations of interest are captured by the X-ray inspection unit. At block 1116, the imagery is analyzed (e.g., by processor(s) 1008), for instance to detect the phase transformation point and/or evaluate intermetallic thicknesses. Some or all of the analysis may occur in real time as the reflow profile is being run, or afterwards upon completion of reflow. And at block 1118, the reflow profile may be updated as needed based on the analysis. At this point, the updated reflow profile may be reloaded as shown by the dashed arrow to block 1102.

The foregoing examples are not mutually exclusive, but may be implemented in various combinations to achieve unique advantages. As these and other variations and combinations of the features discussed above can be utilized without departing from the subject matter defined by the claims, the foregoing description of the embodiments should be taken by way of illustration rather than by way of limitation of the subject matter defined by the claims. In addition, the provision of the examples described herein, as well as clauses phrased as “such as,” “including” and the like, should not be interpreted as limiting the subject matter of the claims to the specific examples; rather, the examples are intended to illustrate only one of many possible embodiments. Further, the same reference numbers in different drawings can identify the same or similar elements.

1. A reflow method, comprising:
loading a reflow profile into a processing system of a reflow module;
installing a printed circuit board assembly within a reflow chamber of the reflow module, the reflow chamber including one or more temperature sensors arranged to detect temperature within the reflow chamber along one or more selected regions of interest of the printed circuit board assembly;
arranging an x-ray inspection unit of the reflow module with respect to the reflow chamber, the x-ray inspection unit being configured to capture x-ray imagery of the printed circuit board assembly during reflow of solder;
heating the printed circuit board assembly according to the reflow profile;
during heating of the printed circuit board assembly, capturing x-ray imagery of selected areas of the printed circuit board assembly and capturing temperature data along the one or more selected regions of interest of the printed circuit board assembly; and
analyzing the captured x-ray imagery and the captured temperature data to identify information about the solder during reflow.

2. The method of claim 1, further comprising:
modifying the reflow profile based on the analyzing; and
heating another printed circuit board assembly according to the modified reflow profile.

3. The method of claim 2, wherein modifying the reflow profile includes modifying one or more of peak temperature, overall reflow time, time above liquidus, ramp up rate, or ramp down rate.

4. The method of claim 1, wherein analyzing the captured x-ray imagery and the captured temperature data is done in real time during reflow.

5. The method of claim 1, wherein the information about the solder during reflow includes one or more of peak temperature, time above liquidus (TAL), ramp up rate or ramp down rates.

6. The method of claim 1, wherein information about the solder during reflow includes intermetallic connection thickness information for one or more solder joints of the printed circuit board assembly.

7. The method of claim 6, wherein when the intermetallic connection thickness information indicates a solder joint thickness below a threshold value, the method further includes modifying the reflow profile.

8. The method of claim 6, further comprising evaluating the intermetallic connection thickness information in view of possible deformation under stratospheric conditions.

9. The method of claim 1, wherein analyzing the captured x-ray imagery and the captured temperature data includes detecting a phase transformation point for the solder.

10. The method of claim 1, further comprising selecting a different solder material or component alloy based on the analyzing.

11. The method of claim 1, wherein the x-ray imagery comprises still images or video imagery.

12. The method of claim 1, wherein arranging the x-ray inspection unit of the reflow module with respect to the reflow chamber comprises placing the reflow module within the x-ray inspection unit.

13. The method of claim 12, further comprising setting the x-ray inspection unit in vacuum after placing the reflow module within the x-ray inspection unit.

14. The method of claim 1, wherein analyzing the captured x-ray imagery and the captured temperature data includes evaluating the captured x-ray imagery and the captured temperature to identify a point in time at which a phase transformation point of the solder occurs.

15. The method of claim 1, further comprising arranging the one or more temperature sensors to detect temperature within the reflow chamber along the one or more selected regions of interest of the printed circuit board assembly based on positions of one or more components on the printed circuit board assembly.

16. The method of claim 1, wherein arranging the x-ray inspection unit includes orienting one or more x-ray imagers to capture the imagery along the one or more selected regions of interest of the printed circuit board assembly based on positions of one or more components on the printed circuit board assembly.

17. The method of claim 1, wherein analyzing the captured x-ray imagery and the captured temperature data to identify information about the solder during reflow including identifying one or more solder defects along the printed circuit board assembly.

18. A printed circuit board assembly fabricated using the reflow method of claim 1.

19. A communication system, comprising one or more printed circuit board assemblies fabricated by the method of claim 1.

20. The communication system of claim 19, wherein the communication system further includes a high altitude platform configured to operate in the stratosphere.
